(19) United States
(12) Patent Application Publication
(10) Pub. No.: US 2011/0111055 A1
(43) Pub. Date: May 12, 2011

(54) NUTRITIONAL SUPPLEMENTS FOR RELIEF OF DRY EYE

(75) Inventor: John C. Lang, Cedar Hill, TX (US)

(73) Assignee: ALCON RESEARCH, LTD., Fort Worth, TX (US)

(21) Appl. No.: 12/941,305

(22) Filed: Nov. 8, 2010

Related U.S. Application Data
(60) Provisional application No. 61/258,867, filed on Nov. 6, 2009.

(51) Int. Cl. A61K 33/34; A61K 31/35; A61K 31/355

(52) U.S. Cl. 424/638; 514/456; 514/458

(57) ABSTRACT

The present invention provides improved dietary supplements and methods for alleviating the symptoms of dry eye. The dietary supplements of the invention contain omega-3 fatty acids and flavonoids.

### BACKGROUND OF THE INVENTION

This application claims priority under 35 U.S.C. §119 to U.S. Provisional Patent Application No. 61/258,867 filed Nov. 6, 2009, the entire contents of which are incorporated herein by reference.

1. Field of the Invention

The present invention generally relates to nutritional methods and compositions for alleviating eye diseases and, more specifically, to improved methods and compositions for the is relief of symptoms of dry eye.

2. Description of the Related Art

Dry eye, also referred to as keratoconjunctivitis sicca, is a common ophthalmological disorder affecting millions of persons each year. The condition is particularly widespread among post-menopausal women due to hormonal changes following the cessation of fertility. Dry eye may afflict an individual with varying severity. In mild cases, a patient may experience burning, a feeling of dryness, and persistent irritation such as is often caused by small bodies lodging between the eye lid and the eye surface. In severe cases, vision may be substantially impaired. Other diseases, such as Sjogren's disease and cicatricial pemphigoid, may also lead to dry eye conditions. Transient symptoms of dry eye associated with refractive surgery have been reported to last in some cases from six weeks to six months or more following surgery.

Although it appears that dry eye may result from a number of unrelated pathogenic causes, all presentations of the complication share a common effect, that is the breakdown of the pre-ocular tear film, which results in exposure of the ocular surface, dehydration, and cytokine production resulting in many of the symptoms outlined above (Lemp, Report of the National Eye Institute/Industry Workshop on Clinical Trials in Dry Eyes, The CLAO Journal, volume 21, number 4, pages 221-231 (1995)).

Practitioners have taken several approaches to the treatment of dry eye. One common approach has been to supplement and stabilize the ocular tear film using so-called artificial tears instilled throughout the day. Other approaches include the use of ocular inserts that provide a tear substitute or stimulation of endogenous tear production.

Examples of the tear substitution approach include the use of buffered, isotonic saline solutions, aqueous solutions containing water soluble polymers that render the solutions more viscous and thus less easily shed by the eye. Tear reconstitution is also attempted by providing one or more components of the tear film such as phospholipids and oils. Phospholipid compositions have been shown to be useful in treating dry eye; see, e.g., McCulley and Shine, Tear film structure and dry eye, Contactologia, volume 20(4), pages 145-49 (1998); and Shine and McCulley, Keratoconjunctivitis sicca associated with meibomian secretion polar lipid abnormality, Archives of Ophthalmology, volume 116(7), pages 849-52 (1998).

Another approach involves the provision of lubricating substances in lieu of artificial tears. For example, U.S. Pat. No. 4,818,537 (Guo) discloses the use of a lubricating, liposome-based composition, and U.S. Pat. No. 5,800,807 (Hu et al.) discloses compositions containing glycerin and propylene glycol for treating dry eye.

Although these approaches have met with some success, problems in the treatment of dry eye nevertheless remain, since the use of tear substitutes, while temporarily effective, generally requires repeated application over the course of a patient's waking hours. It is not uncommon for a patient to have to apply artificial tear solution ten to twenty times over the course of the day. Such an undertaking is not only cumbersome and time consuming, but is also potentially very expensive.

Aside from efforts described above, which are directed primarily to the palliative alleviation of symptoms associated with dry eye, methods and compositions directed to treatment of the physiological conditions that cause such symptoms have also been pursued. For example, U.S. Pat. No. 5,041,434 (Lubkin) discloses the use of sex steroids, such as conjugated estrogens, to treat dry eye conditions in post-menopausal women; U.S. Pat. No. 5,290,572 (MacKeen) discloses the use of finely divided calcium ion compositions to stimulate pre-ocular tear film production.

Such efforts to treat the underlying causes of dry eye have focused on treating inflammation of the relevant ocular tissues and meibomian gland dysfunction. The use of various types of agents for such treatment of dry eye patients has been disclosed, including steroids (e.g., U.S. Pat. No. 5,958,912; Marsh et al., Topical nonpreserved methylprednisolone therapy for keratoconjunctivitis sicca in Sjogren syndrome, Ophthalmology, 106(4): 811-816 (1999); and Pflugfelder et al., U.S. Pat. No. 6,153,607), cytokine release inhibitors (Yanni, J. M.; et. al. WO 00/03705 A1), cyclosporine A (Tauber, J. Adv. Exp. Med. Biol. 1998, 438 (Lacrimal Gland, Tear Film, and Dry Eye Syndromes 2), 969), and mucosecretatogues, such as 15-HETE (Yanni et. al., U.S. Pat. No. 5,696,166).

There are also commercially available nutritional products claiming benefit for dry eye. Ingestion of dietary supplements provides a means of alleviating the symptoms of dry eye while avoiding the need for multiple re-applications of lubricant drops throughout the day. Dietary supplements are taken for a variety of reasons including the improvement of vision or prophylaxis against vision loss. An example of a set of dietary supplements useful in promoting healthy eyes (but not for alleviating the symptoms of dry eye) are the ICAPS® Dietary Supplements (Alcon Laboratories, Inc., Fort Worth, Tex.). Dietary supplements are generally in the form of powders, tablets, chewable tablets, capsules, gel-caps or liquid-fill softgels and comprise a variety of vitamins, minerals, and herbal or other organic constituents. Some dietary supplements are formulated with beadlets. There do not appear to be any commercially available dietary supplements containing the narrow selection of ingredients described herein for the alleviation of symptoms of dry eye.

### SUMMARY OF THE INVENTION

The present invention overcomes these and other drawbacks of the prior art by providing a dietary supplement containing a unique combination of vitamins, minerals, and essential nutrients useful for alleviating the symptoms of dry eye. In particular, the improved formulations described herein comprise essential omega-3 fatty acids in conjunction with genistein. Such improved formulations may additionally provide up to 2000 mg of EPA/DHA, up to 200 mg alpha-linolenic acid, up to 60 mg vitamin C, up to 200 mg d-alpha-tocopheryl acetate, up to 150 mcg vitamin D, up to 30 mg zinc, up to 100 mg copper, up to 70 mcg selenium, up to 4 mg manganese, up to 100 mg genistein, up to 25 mg lycopene, and up to 25 mg carnosol or carnosic acid. As use herein, the term “up to” encompasses no amount of the referenced ingredient. For example, “up to 600 mg of EPA/DHA” means from 0 mg to 600 mg of EPA/DHA. Preferred formulations contain about 300 mg EPA/DHA as ethyl esters, about 50 mg alpha-linolenic acid as ethyl ester, about 30 mg vitamin C, about 50 mg d-alpha-tocopheryl acetate, about 5 mcg vitamin D, about 15 mg zinc, about 1 mg copper, about 35 mcg selenium, about 2 mg manganese, about 50 mg genistein, about 10 mg lycopene, and about 10 mg carnosol and carnosic acid as rosemary oil or rosemary extract.

### DETAILED DESCRIPTION PREFERRED EMBODIMENTS

According to the present invention, the elements of the composition are directed toward alleviating the symptoms of dry eye by providing essential omega-3 fatty acids in conjunction with the phytochemical genistein.

Dry eye often is accompanied by inflammation. Blepharitis, for example, is frequently associated with the burning and irritation of dry eye. Proinflammatory cytokines have been observed in some patients with dry eye, and are especially elevated in the more severe types of dry eye, such as Sjögren's syndrome.

Omega-3 fatty acids, found naturally and in abundance in tissue of cold water fish, are also abundant in the optic discs of photoreceptors in human retina. Epidemiologically, it has been found that the prevalence of AMD is higher for individuals with diets depleted in omega-3 fatty acids, that is, that the amount of omega-3 in the diet correlates inversely with the prevalence of AMD (Seddon and Willett el al.). It is known that essential omega-3 fatty acids, such as the C₂₀ eicosapentaenoic acid (EPA) and the C₂₂ docosahexaenoic acid (DHA), may assist in regulating the level of proinflammatory prostaglandins by competing with arachidonic acid as substrates for cyclooxygenases and lipoxygenases. These important fish oil-derived essential nutrients are believed to reduce the incidence and progression of AMD, for which an inflammatory involvement has been attributed. Polyene omega-3 fatty acids are readily oxidized. The products of such oxidation are easily functionalized in vivo, especially by cytochrome enzymes. Some omega-3 fatty acids are integral to the structure of membranes. Others appear to function as ligands for transcription factors or nuclear receptors, serving as a messenger to either activate or inactivate nuclear activity. Therefore, members of the class of omega-3 fatty acids may provide added duration of effect, or impart stability, in the dietary supplements of the invention, while avoiding the unpleasant “fishy” odor that can accompany this class of essential fat.

The two predominant omega-3 fatty acids, conjugated fatty acids, important in eye health are DHA and EPA. The term “DHA” as used herein refers to either of these two predominant omega-3 fatty acids or to a mixture of the two; that is, when the term “DHA” is used, the skilled artisan would understand that either DHA, EPA, or a mixture of EPA and DHA could be used in that instance. The preferred ratio of EPA to DHA when a mixture is used is 0.8:0.2 to 0.2:0.8, EPA:DHA. While docosahexaenoic has been made available from fermentation and biotechnology sources, the preferred blend is usually harvested from fish and then purified/deodorized.

Bioflavonoids, or “flavonoids,” are flavone- and isoflavone-like structures found primarily in fruits and vegetables. Flavonoids (polyphenolics readily functionalized in vivo producing metabolites often difficult to track) are well known to serve as both good antioxidants and biological messengers. Other isoflavones, either parent or functionalized, may also function as nuclear messengers regulating transcription or translation and thereby influencing proteins controlling either inflammation or cell secretion as effectively in humans as genistein functions in rats (see e.g., Example 1). Functionalized isoflavones might be selected for potency or duration of biological effect, stability of a formulated material, or bioavailability. Bioflavonoids are commercially available or may be synthesized by methods known in the art. Examples of bioflavonoids include, but are not limited to, quercetin, acacetin, liquiritin, rutin, taxifolin, nobiletin, tangeretin, apigenin, chyrsin, myricetin, genistein, daidzein, luteolin, naringenin, and kaempferol, and their derivatives, such as the corresponding methoxy-substituted analogs. The bioflavonoids may be useful in nutritional health as modulators of the rates of in vivo enzyme-mediated reactions.

It is believed that the combination of ingredients (i.e., omega-3 fatty acids and flavonoids) in the formulations and dietary supplements of the present invention will provide not only an additive, but also a synergistic effect to the dietary supplements containing them. The combination of flavonoid with omega-3 fatty acids should be of greater benefit than either alone for the treatment of dry eye, potentially influencing the level of inflammation, dryness, and Meibomian secretion.

In certain embodiments, the dietary supplements as described herein contain at least one omega-3 fatty acid and at least one flavonoid compound. Preferred omega-3 fatty acids for use in the dietary supplements of the present invention include Ropufa 75 N-3 EE (a proprietary blend of omega-3 fatty acids produced by DSM). Preferred flavonoids for use in the dietary supplements of the present invention include quercetin, acacetin, liquiritin, rutin, taxifolin, nobiletin, tangeretin, apigenin, chyrsin, myricetin, genistein, daidzein, luteolin, naringenin, and kaempferol, and their derivatives, such as the corresponding methoxy-substituted analogs.

The anticipated dosage form will be a softgel. The preferred dosing regimen will be ingestion of two to three softgel capsules per day of approximately one gram (1 g) total weight each, containing a blend having about 60% omega-3 and about 40% flavonoid, by weight. The most preferred formulations of the present invention include those in examples 1-4.

The formulations of the present invention may also contain one or more additional antioxidants. The antioxidants can be hydrophobic or hydrophilic. The antioxidants serve to inhibit the oxidative, photochemical and/or thermal degradation of the carotenoid components. Since antioxidants are also thought to be useful in nutritional health, they may also provide some nutritional benefit to the host. In general, the antioxidants will be natural antioxidants or agents derived therefrom. Examples of natural antioxidants and related derivatives include, but are not limited to, vitamin E and related derivatives, such as tocotrienols, alpha-, beta-, gamma-, delta- and epsilon-tocopherol, and their derivatives, such as the corresponding acetates, succinates; Vitamin C and related derivatives, e.g., ascorbyl palmitate; and natural oils, such as oil of rosemary. Preferred formulations will contain one or more hydrophobic antioxidants. The amount of antioxidant(s) contained in the formulation will be an amount effective to inhibit or reduce the oxidative, photochemical and/or thermal degradation of the carotenoid components. Such an amount is referred to herein as “an effective amount of one or more antioxidants.” In general, such an amount will range from about 0.1 to 10 times the amount of the xanthophyll and carotene/retinoid components and any other chemically sensitive components present, e.g., bioflavonoids. Preferred formulations, which will generally comprise about 0.5-25% w/w of carotenoids alone, or including bioflavonoids, will contain about 2 to 10% w/w of antioxidant. The antioxidants may be combined with designated nutrients in isolated reservoirs of cobeadlets before incorporation into the dosage form. Cobeadlets such as those described in U.S. Pat. Nos. 6,582,721, and 6,716,447, and in U.S. Patent Application Nos. 2005/0106272, and 2005/0147698, all of which are incorporated herein by reference, would be useful in the formulations of the present invention.

The formulations will also comprise one or more solidifying, bulking and agglomerating agents (collectively referred to herein as “solidifying agent(s)”). The solidifying agent(s) are used both in tableting and in generating solid-like carriers such as beadlets, capable of transforming oils into stable agglomerates suitable for granulation, blending, and compression required for tableting. Examples of solidifying agents useful in the preparation of the formulations include, but are not limited to, sucrose, glucose, fructose, starches (e.g., corn starch), syrups (e.g., corn syrup), and ionic and nonionic polymers including, but not limited to, PEGs and other poly ether-like alkoxy cellulosics (HPMC), gellan, carrageenans, Eucheuma gelatenae, guar, hyaluronates, alginates, chondroitin sulfate, pectins, and proteins, (e.g., collagen or their hydrolyzed products (e.g., gelatins or polypeptides)). Other solidifying agents known to those skilled in the art of dietary supplement preparation may also be used in the preparation of the formulations of the present invention. The amount of solidifying agent(s) will vary, depending on the other components contained in the formulation, but will generally comprise the majority weight and volume of the dietary supplement.

The dosage form of the dietary supplements described herein is preferably a tablet, caplet or softgel form for oral administration, with the patient taking one to four doses taken once or twice a day. The present invention, however, contemplates that the preferred total dosage can be administered as a single dose or other multiple part dosages. The composition may also be of the timed-release or delayed-release types. Further, for oral administration, the present composition may be in capsules, lacquered tablets, unlacquered tablets, softgels, or blends of controlled release powders, prepared according to well-known methods. In accordance with the preferred multiple dosages described above, each tablet, caplet, or softgel is preferably composed approximately as follows:

Other oils may be present in the formulations of the present invention. The formulations will typically comprise an amount of vegetable oils or oleoresins, since the separate carotene/retinoid and/or xanthophyll components to be added to the formulations are generally commercially available as a diluted vegetable oil or oil suspension, or as an oleoresin extract. Such an amount of oil/oleoresin typically ranges from about 1 to 100 times the xanthophyll or carotene content in the formulation. For example, a xanthophyll extract to be included in a dietary supplement may contain 20% w/w lutein, 2% w/w zeaxanthin and 78% vegetable oil/oleoresin. Other oils may also be included in the formulations.

The formulations of the present invention may also comprise additional excipients useful in preparing and finishing the dietary supplements. Such excipients may include timed-release polymer coating agents useful in prolonging dissolution of the formulation in the digestive tract. Examples of such polymers include, but are not limited to ionic and nonionic polymers, such as PEGs and other poly ether-like alkoxy cellulosics (HPMC), gellan, carrageenans, Eucheuma gelatenae, starch, hyaluronates, chondroitin sulfate, pectins, and proteins, e.g., collagen. Since the xanthophyll/carotenes are highly pigmented, coating technology may be applied to the dietary supplement in order to provide a dietary supplement of uniform color. Examples of color coating agents may include, but are not limited to, polymers, colorants, sealants and surface active agents including, not limited to, fatty acids and esters, di- and triglycerides, phospholipids including mono- and di-alkyl glyceryl phosphates, nonionic agents (sugars, polysaccharides, e.g., HPMC and polysorbate 80) and ionic agents.

The above-described ingredients contained in the formulations may, in some cases, form microspheres within the dietary supplement. The dietary supplements may be of various size and shape.

The dietary supplements may be manufactured using a number of techniques known in the art. The ingredients described herein are preferably present in the dietary supplements of the invention in an amount sufficient to provide the daily dosage (amount consumed per day) when the recommended number of dietary supplements is ingested per day.

In some dosage forms, such as softgels, the use of concentrated oil phases of nutrients is desirable. These may be combined into a composite flowable core and concurrently protected with the aid of common diluents and antioxidants.

The following Examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

The following table provides examples of omega-3 fatty acids and flavonoids considered to be useful in the compositions and methods of the present invention. The skilled artisan will understand that the agents provided below can be included in the dietary supplements of the invention in any amount within the range provided and, further, that any combination of the primary and secondary ingredients listed can be provided in the dietary supplements.

| | Concentration Range | | Daily |
|---|---|---|---|
| Ingredient | (mg/capsule) | | Dose |
| Primary | | | |
| Omega-3 FA's | 0-500 | mg/cap | 1-3 caps |
| EPA | | | |
| DHA | | | |
| Alpha-Linolenic | | | |
| Omega-3 FA's | 0-500 | mg/cap | 1-3 caps |
| Gamma-Linolenic | | | |
| Vitamin D3 | 0-25 | mg/cap | 1-3 caps |
| Isoflavones & Flavones | 0-500 | mg/cap | 1-3 caps |
| Genistein | | | |
| Quercetin | | | |
| Catechins | 0-500 | mg/cap | 1-3 caps |
| Epigallocatechingallate | | | |
| Rosemary/components | 0-100 | mg/cap | 1-3 caps |
| Camosic Acid | | | |
| Carnosol | | | |
| Rosmarinic acid | | | |
| Curcumin | 0-100 | mg/cap | 1-3 caps |
| Secondary | | | |
| Vitamin C | 0-300 | mg/cap | 1-3 caps |
| Vitamin E | 0-500 | mg/cap | 1-3 caps |
| Polymer for | 0-300 | mg/cap | 1-3 caps |

Bioavailability
HPMC
Guar
Carrageenan

Further examples of preferred formulations according to the present invention are provided in the following examples.

### EXAMPLE 2

Amount to be delivered in 2 Capsules/day

| | | Typical Daily | |
|---|---|---|---|
| Ingredients | Units | Amount | Range |
| Omega-3 Fatty Acids | mg | 800 | 0-2,000 |
| Vitamin E | mg | 50 | 0-200 |
| Vitamin D | mcg | 50 | 0-150 |
| Rosemary Extract | mg | 10 | 0-25 |

### EXAMPLE 3

Amount to be delivered in 2 Capsules/day

| | | Typical Daily | |
|---|---|---|---|
| Ingredients | Units | Amount | Range |
| EPA/DHA | mg | 600 | 0-2,000 |
| d-alpha-Tocopheryl Acetate | mg | 50 | 0-200 |
| Vitamin D | mcg | 50 | 0-150 |
| Rosemary Extract | mg | 10 | 0-25 |

### EXAMPLE 4

Amount to be delivered in 2 Capsules/day

| | | Typical Daily | |
|---|---|---|---|
| Ingredients | Units | Amount | Range |
| EPA/DHA | mg | 600 | 0-2,000 |
| ALA (alpha-Linolenic Acid) | mg | 50 | 0-200 |
| d-alpha-Tocopheryl Acetate | mg | 50 | 0-200 |
| Vitamin D | mcg | 50 | 0-150 |
| Rosemary Extract | mg | 10 | 0-25 |

### EXAMPLE 5

Amount to be delivered in 2 Capsules/day

| | | Typical Daily | |
|---|---|---|---|
| Ingredients | Units | Amount | Range |
| EPA/DHA Ethyl Esters | mg | 600 | 0-2,000 |
| ALA (alpha-Linolenic Acid) | mg | 50 | 0-200 |
| as Ethyl Ester | | | |
| d-alpha-Tocopheryl Acetate | mg | 50 | 0-200 |
| Vitamin D | mcg | 50 | 0-150 |
| Rosemary Extract | mg | 10 | 0-25 |

### EXAMPLE 6

Amount to be delivered in 2 Capsules/day

| | | Typical Daily | |
|---|---|---|---|
| Ingredients | Units | Amount | Range |
| EPA/DHA Ethyl Esters | mg | 600 | 0-2,000 |
| ALA (alpha-Linolenic Acid) | mg | 50 | 0-200 |
| as Ethyl Ester | | | |
| d-alpha-Tocopheryl Acetate | mg | 50 | 0-200 |
| Vitamin D | mcg | 50 | 0-150 |
| Genistein | mg | 50 | 0-100 |
| Rosemary Extract | mg | 10 | 0-25 |

### EXAMPLE 7

Amount to be delivered in 1 Capsule/day

| | | Typical Daily | |
|---|---|---|---|
| Ingredients | Units | Amount | Range |
| EPA/DHA Ethyl Esters | mg | 300 | 0-600 |
| ALA (alpha-Linolenic Acid) | mg | 50 | 0-200 |
| as Ethyl Ester | | | |
| d-alpha-Tocopheryl Acetate | mg | 50 | 0-200 |
| Vitamin D | mcg | 50 | 0-150 |
| Genistein | mg | 50 | 0-100 |
| Rosemary Extract | mg | 10 | 0-25 |

### EXAMPLE 8

Amount to be delivered in 1 Capsule/day

| | | Typical Daily | |
|---|---|---|---|
| Ingredients | Units | Amount | Range |
| EPA/DHA Ethyl Esters | mg | 300 | 0-600 |
| ALA (alpha-Linolenic Acid) | mg | 50 | 0-200 |
| as Ethyl Ester | | | |
| Vitamin C | mg | 30 | 0-60 |
| d-alpha-Tocopheryl Acetate | mg | 50 | 0-200 |
| Vitamin D | mcg | 5 | 0-150 |
| Zinc | mg | 15 | 0-30 |
| Copper | mg | 1 | 0-100 |
| Silenium | mcg | 35 | 0-70 |
| Manganese | mg | 2 | 0-4 |
| Genistein | mg | 50 | 0-100 |
| Lycopene | mg | 10 | 0-25 |
| Rosemary Extract | mg | 10 | 0-25 |

### EXAMPLE 9

Amount to be delivered in 1 Capsule/day

| | | Typical Daily | |
|---|---|---|---|
| Ingredients | Units | Amount | Range |
| EPA/DHA Ethyl Esters | mg | 300 | 0-600 |
| ALA (alpha-Linolenic Acid) | mg | 50 | 0-200 |
| as Ethyl Ester | | | |
| Vitamin C | mg | 30 | 0-60 |
| d-alpha-Tocopheryl Acetate | mg | 50 | 0-200 |
| Vitamin D | mcg | 5 | 0-150 |
| Zinc | mg | 15 | 0-30 |
| Copper | mg | 1 | 0-100 |
| Selenium | mcg | 35 | 0-70 |
| Manganese | mg | 2 | 0-4 |
| Genistein | mg | 50 | 0-100 |
| Lycopene | mg | 10 | 0-25 |
| Carnosol & Carnosic | mg | 10 | 0-25 |

### EXAMPLE 10

Amount to be delivered in 1 Capsule or 1 Tablet/day

| | | Typical Daily | |
|---|---|---|---|
| Ingredients | Units | Amount | Range |
| EPA/DHA Ethyl Esters | mg | 300 | 0-600 |
| ALA (alpha-Linolenic Acid) | mg | 50 | 0-200 |
| as Ethyl Ester | | | |
| Vitamin C* | mg | 30 | 0-60 |
| d-alpha-Tocopheryl Acetate | mg | 50 | 0-200 |
| Vitamin D | mcg | 5 | 0-150 |
| Zinc* | mg | 15 | 0-30 |
| Copper* | mg | 1 | 0-100 |
| Selenium* | mcg | 35 | 0-70 |
| Manganese* | mg | 2 | 0-4 |
| Genistein | mg | 50 | 0-100 |
| Lycopene | mg | 10 | 0-25 |
| Carnosol & Carnosic acid** | mg | 10 | 0-25 |

*May be omitted from the Tablet form.
**May be provided as rosemary oil.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

United States Patents and Published Applications
U.S. Pat. No. 3,998,753
U.S. Pat. No. 4,254,100
U.S. Pat. No. 4,657,928
U.S. Pat. No. 4,670,247
U.S. Pat. No. 6,582,721
U.S. Pat. No. 6,716,447
20030064133
20050106272
20050147698

Books
Berman, BIOCHEMISTRY OF THE EYE, (Plenum, 1991).
Chaney TEXTBOOK OF BIOCHEMISTRY WITH CLINICAL CORRELATIONS, John Wiley & Sons, pp. 970-1 (1986)

Other Publications
Beem J BIOL CHEM 249:7298 (1974)

Bernstein et al., Retinal Tubulin Binds Macular Carotenoids, INV OPHTHAL & VIS SCI 38(1):167-175 (1997).
Bernstein et at Identification and Characterization of Pi Isoform of Glutathione S-Transferase (GSTP1) as a Zeaxanthin-binding Protein in the Macula of the Eye, J. BIOL. CHEM 279(47):49447-49454 (2004)
Bone and Landrum et al. Analysis of Macular Pigment by HPLC: Retinal Distribution and Age Study INV. OPHTH VIS SCI 29:843-849 (1988).
Bone and Landrum et al. Macular Pigment in Donor Eyes with and without AMD: a Case-control Study, INV. OPHTH VIS SCI 42:235-240 (2001).
Chandler et al., J. BIOL. CHEM 261:928-33 (1986)
Chatterjee ARCH, OPHTHALMOL 56:756-60 (1956)
Fischer J NUTRITION 113:462-9 (1983)
Hammond et al., Sex differences in macular pigment optical density: relation to plasma carotenoid concentrations and dietary patterns, VISION RESEARCH 36:2001-2012 (1996a).
Hammond et al., Cigarette smoking and retinal carotenoids: implications for age-related macular degeneration, VISION RESEARCH 36:3003-3009 (1996b).
Hammond et al., Dietary modification of human macular pigment density, INV OPHTHAL & VIS SCI 38(9):1795-1801 (1997).
Handelman et al., Biological control of primate macular pigment: biochemical and densitometric studies, INV OPHTHAL & VIS SCI 32(2):257-267 (1991).
Harris, NATURE 132:27-8 (1993)
Hooper, JAMA 244:1960-1 (1980)
Jacques et al., Antioxidant status in persons with and without senile cataract, ARCH. OPHTHALM. 106:337 (1988).
Karcioglu SURV OPHTHALMOL 27:114-22 (1982)
Krinsky et al., Biologic mechanism of the protective role of lutein and zeaxanthin in the eye, ANNUAL REV NUTR 23:171-201 (2003)
Leure-duPree, RETINA 2:294-302 (1982a)
Leure-duPree, INVEST OPHTHALMOL VIS SCI 23:425-34 (1982b)
Machlin et al., Free radical tissue damage: protective role of antioxidant nutrients, FASEB J 1:441-445 (1987).
Newsome, D. A., Oral zinc in macular degeneration, ARCH. OPHTHALMOL. 106:192-198 (1988).
Ohrloff GRAEFE'S ARCH CLIN EXP OPHTHALMOL 222:79-81 (1984)
Orten, HUMAN BIOCHEMISTRY 10^{th} Edition, CV Mosby Co., p. 756 (1982)
Pennington J AM DIETETIC ASSOC 86:876-91 (1986)
Purcell ARCH, OPHTHALMOL 51:1-6 (1968)
Ringvold ACTA, OPHTHALMOLOGICA 63:227-80 (1985)
Russell ANN INT MED 99:227-39 (1983)
Seddon et al., Dietary carotenoids, vitamins a, c and e, and advanced age-related macular degeneration, JAMA 272(8):1413-1420 (1994).
Seddon and Willett et al., Prospective study of dietary fat and the risk of age-related macular degeneration, AM J CLIN NUTR 73:209-218 (2001).
Snodderly, Evidence for protection against age-related macular degeneration by carotenoids and antioxidant vitamins AM J CLIN NUTR 62 (suppl):1448S-1461S (1995).
Spector et al., EXP. EYE RES. 33:673 (1981).
Swanson BIOCHEM BIPHY RES COMM 45:1488-96 (1971)
Taylor, NUTRITIONAL AND ENVIRONMENTAL INFLUENCES ON THE EYE, (CRC, 1999).
Van Campen J NUTRITION 97:104-8 (1970)
Varma OPHTHALMIC RES 9:421-31 (1977)
Wagner GERIATRICS 40:111-25 (1985)
Williams PEDIAT RES 1:823 (1977)

We claim:

1. A dietary supplement for alleviating the symptoms of dry eye comprising at least one omega-3 fatty acid and at least one flavonoid.

2. The dietary supplement of claim 1, wherein the omega-3 fatty acid is ROPUFA 75 N-3 EE.

3. The dietary supplement of claim 1, wherein the flavonoid is selected from the group consisting of quercetin, acacetin, liquiritin, rutin, taxifolin, nobiletin, tangeretin, apigenin, chyrsin, myricetin, genistein, daidzein, luteolin, naringenin, and kaempferol, and their derivatives.

4. The dietary supplement of claim 3, wherein the flavonoid is genistein.

5. The dietary supplement of claim 1, wherein the dietary supplement is in the form of a softgel having a total weight of one gram (1 g), and wherein the concentration of the omega-3 fatty acid is about 60% by weight and the concentration of the flavonoid is about 40% by weight.

6. The dietary supplement of claim 1, further comprising at least one antioxidant.

7. The dietary supplement of claim 6, wherein the antioxidant is selected from the group consisting of vitamin E and related derivatives; Vitamin C and related derivatives; and natural oils.

8. A dietary supplement for alleviating the symptoms of dry eye comprising up to 600 mg of EPA/DHA, up to 200 mg alpha-linolenic acid, up to 60 mg vitamin C, up to 200 mg d-alpha-tocopheryl acetate, up to 150 mcg vitamin D, up to 30 mg zinc, up to 100 mg copper, up to 70 mcg selenium, up to 4 mg manganese, up to 100 mg genistein, up to 25 mg lycopene, and up to 25 mg carnosol or carnosic acid.

9. The dietary supplement of claim 8, comprising about 300 mg EPA/DHA as ethyl esters, about 50 mg alpha-linolenic acid as ethyl ester, about 30 mg vitamin C, about 50 mg d-alpha-tocopheryl acetate, about 5 mcg vitamin D, about 15 mg zinc, about 1 mg copper, about 35 mcg selenium, about 2 mg manganese, about 50 mg genistein, about 10 mg lycopene, and about 10 mg carnosol and carnosic acid.

10. The dietary supplement of claim 9, wherein the carnosol and carnosic acid are provided as rosemary oil.

11. The dietary supplement of claim 9, wherein the dietary supplement is a capsule.
